(19) United States
(12) Patent Application Publication
(10) Pub. No.: US 2025/0345297 A1
(43) Pub. Date: Nov. 13, 2025

(54) ANTI-ARRHYTHMIC COMPOSITIONS AND METHODS

(71) Applicant: AltaThera Pharmaceuticals LLC, Chicago, IL (US)

(72) Inventors: Brandon Ira Kashfian, Chicago, IL (US); Jodi Devlin, Chicago, IL (US)

(21) Appl. No.: 19/277,199

(22) Filed: Jul. 22, 2025

Related U.S. Application Data
(62) Division of application No. 17/892,301, filed on Aug. 22, 2022, now Pat. No. 12,396,970.
Application No. 17/892,301 is a continuation-in-part of application No. 17/566,840, filed on Dec. 31, 2021, now Pat. No. 11,610,660.
(60) Provisional application No. 63/235,500, filed on Aug. 20, 2021.
(60) Provisional application No. 63/235,500, filed on Aug. 20, 2021.
(60) Provisional application No. 63/276,947, filed on Nov. 8, 2021.
(60) Provisional application No. 63/276,947, filed on Nov. 8, 2021.
(60) Provisional application No. 63/331,905, filed on Apr. 18, 2022.
(60) Provisional application No. 63/334,267, filed on Apr. 25, 2022.
(60) Provisional application No. 63/340,581, filed on May 11, 2022.
(60) Provisional application No. 63/344,154, filed on May 20, 2022.
(60) Provisional application No. 63/345,068, filed on May 24, 2022.

(51) Int. Cl. A61K 31/18; A61K 9/00; A61P 9/06

(52) U.S. Cl. CPC A61K 31/18; A61P 9/06; A61K 9/0019

(57) ABSTRACT

Methods of administering an anti-arrhythmic, such as dofetilide, to a patient in an amount effective for treating a cardiovascular condition are described. The drug can be administered intravenously for at least one hour. A loading dose of 0.1 to 12 μg/kg bodyweight over a duration of up to 60 minutes can be administered and/or a maintenance dose of 0.1 to 10 μg/kg/hr can be administered intravenously over a duration of at least 1 hour, optionally alternatively or in addition wherein the amount of the loading dose and/or the IV maintenance dose is in the range of about ±50% of a maintenance dofetilide dose. The cardiovascular condition can include atrial fibrillation or flutter, ventricular tachycardia, hemodynamically stable or unstable ventricular tachycardia, paroxysmal atrial fibrillation, ventricular fibrillation, paroxysmal supraventricular tachycardia, heart failure, coronary artery disease, or pulmonary artery hypertension. A patient's QT interval and/or a creatinine clearance can be measured, and the effective amount can be selected based on either or both of the QT interval or the creatinine clearance measurements.

### CROSS-REFERENCE TO RELATED APPLICATIONS

This application is a Continuation application of U.S. application Ser. No. 17/892,301 filed Aug. 22, 2022, which application relies on the disclosure of and claims priority to and the benefit of the filing date of U.S. Provisional Application Nos. 63/235,500 filed Aug. 20, 2021, 63/276,947 filed Nov. 8, 2021, 63/331,905 filed Apr. 18, 2022, 63/334,267 filed Apr. 25, 2022, 63/340,581 filed May 11, 2022, 63/344,154 filed May 20, 2022, and 63/345,068 filed May 24, 2022. The '301 application is a Continuation-in-Part application of U.S. application Ser. No. 17/566,840 filed Dec. 31, 2021, which application issued as U.S. Pat. No. 11,610,660 on Mar. 21, 2023, and which application relies on the disclosure of and claims priority to and the benefit of the filing date of U.S. Provisional Application Nos. 63/235,500 filed Aug. 20, 2021 and 63/276,947 filed Nov. 8, 2021, which applications are hereby incorporated by reference herein in their entireties.

### FIELD OF THE INVENTION

The present disclosure is directed to the field of cardiovascular pharmaceutics, more particularly compositions and methods for the treatment of cardiovascular conditions such as arrhythmias with intravenous anti-arrhythmics, such as dofetilide.

### BACKGROUND

Dofetilide is a class III antiarrhythmic agent which acts through blocking cardiac ion channels of the rapid component of the delayed rectifier potassium current Ikr. The agent, a sulfonamide, is approved to treat atrial fibrillation and atrial flutter. Dofetilide normalizes sinus rhythm by prolonging cardiac action potential duration and effective refractory period due to delayed repolarization without affecting conduction velocity. Dofetilide is currently approved in the US for oral administration under the brand name TIKOSYN® (Pfizer Inc., New York, NY). It is not approved for parenteral administration.

### SUMMARY OF THE INVENTION

Described herein are methods of administering one or more anti-arrhythmic, such as a Class I or Class III anti-arrhythmic, for example dofetilide, to a patient in need thereof in an amount effective for treating a cardiovascular condition of the patient. In embodiments, the effective amount of anti-arrhythmic, such as dofetilide, can be administered intravenously over a duration of at least 1 hour, and/or can be administered intravenously as a loading dose of 0.1 to 12 μg/kg bodyweight over a duration of up to about 60 minutes, or more, and/or infused intravenously as a maintenance dose of 0.1 to 10 μg/kg/hr over a duration of at least 1 hour, and/or the dosing can be adjusted for pharmacokinetics of the particular drug being administered, such as for dofetilide or other anti-arrhythmics, including sotalol, amiodarone, ibutilide, dronedarone, procainamide, flecainide, or propafenone.

The cardiovascular condition can include atrial fibrillation, atrial flutter, ventricular tachycardia, hemodynamically stable or unstable ventricular tachycardia, ventricular fibrillation, paroxysmal supraventricular tachycardia, paroxysmal atrial fibrillation, heart failure, coronary artery disease, pulmonary artery hypertension, atrial tachycardia, junctional ectopic tachycardia, or junctional tachycardia. The method can further include measuring a QT interval (or QTc) of the patient before, during, and/or after the administering, and selecting or adjusting the effective amount, the loading dose and/or the maintenance dose based on the QT interval or QTc at any point during administering the protocol. The method can further include measuring a creatinine clearance of the patient before the administering, and selecting or adjusting the effective amount, the loading dose and/or the maintenance dose based on the creatinine clearance.

Compositions for intravenous administration of anti-arrhythmics, including dofetilide, are also provided. The compositions and methods are particularly useful for situations in which oral administration is impractical, such as gastrointestinal conditions affecting drug absorption, recovery from gastrointestinal surgery, and/or intensive care, or where oral administration is not recommended or possible (NPO).

### DETAILED DESCRIPTION

Reference will now be made in detail to various illustrative implementations. It is to be understood that the following discussion of the implementations is not intended to be limiting.

AF is atrial fibrillation.

AFL is atrial flutter.

AF/AFL=atrial fibrillation and/or atrial flutter.

IV is intravenous.

PO means “per os” and refers to an oral dosing regimen.

BID means “bis in die” and means twice a day.

QD means “quaque die” and means once a day.

QID means “quater in die” and means four times a day.

Patient (or subject) refers to a human patient.

BP is blood pressure.

HR is heart rate.

Renally impaired refers to patients/subjects having creatinine clearance rates of ≤60 mL/min, such as ≤30 mL/min.

Cmax ss is the maximal concentration obtained at steady state.

QT is the interval measured from the start of the Q wave or the QRS complex, to the end of the T wave, where the Q wave corresponds to the beginning of ventricular depolarization and the T wave end corresponds to the end of ventricular repolarization.

QTc is the calculated interval that represents the QT interval corrected for heart rate and can be derived by mathematical correlation of the QT interval and the heart rate.

ΔQTc is the difference between a QTc measurement taken prior to the start of treatment and a QTc measured after the start of treatment (e.g., during loading or maintenance).

The terms “treat,” “treating,” and “treatment” refer to any indicia of success in the treatment or amelioration of an injury, disease, or condition, including any objective or subjective parameter such as abatement; remission; diminishing of symptoms or making the injury disease, or condition more tolerable to the subject; slowing in the rate of degeneration or decline; or improving a subject's physical or mental well-being. The treatment or amelioration of symptoms can be based on objective or subjective parameters, including the results of a physical examination, neuropsychiatric examinations, or psychiatric evaluation.

Hospital refers to a medical facility staffed and equipped to provide continuous ECG monitoring and cardiac resuscitation to patients, if needed. Typically, the medical personnel are trained in the management of serious ventricular arrhythmias.

Escalation means increasing the dofetilide dosage of a patient already receiving dofetilide, for example where a subject is currently taking a specific amount of an oral dose and escalation involves administering one or more IV doses to escalate the subject to a higher target oral dose.

The term “about” used herein in the context of quantitative measurements means the indicated amount ±10%. For example, “about 2 mg” can mean 1.8-2.2 mg.

### Formulations

Formulations for parenteral administration of anti-arrhythmics, such as dofetilide, can include sterile aqueous or non-aqueous solutions, suspensions, and emulsions. Formulations for other anti-arrhythmics, including sotalol, amiodarone, ibutilide, dronedarone, procainamide, flecainide, or propafenone can be similar. Parenteral administration of the formulation, if used, is generally characterized by injection and can include subcutaneous, intramuscular, intravenous, intradermal, intrathecal, and epidural administration. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Dofetilide (CAS Registry No. 115256-11-6; Pub Chem ID 71329) has a molecular weight of 441.6 g/mol. According to PubChem, dofetilide is soluble in 0.1M NaOH, acetone, and 0.1M HCl, and very slightly soluble in propan-2-ol. It is also very slightly soluble in water. The molecular structure is shown below:

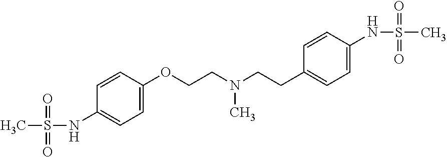

Dofetilide can be formulated in vehicles suitable for intravenous administration, such as those described in Remington: The Science and Practice of Pharmacy (19th ed.) ed. A. R. Gennaro, Mack Publishing Company, Easton, Pa. 1995, which is incorporated by reference herein in its entirety. Typically, an appropriate amount of a pharmaceutically-acceptable salt is used in the formulation to render the formulation isotonic. Examples of the pharmaceutically-acceptable carrier include, but are not limited to, sterile aqueous solutions such as saline, Ringer's solution and dextrose solution. The pH of the solution can be from about 4 to about 8, such as from about 4.5 to about 6.5, about 5 to about 6, or about 7 to about 7.5, and can be adjusted with appropriate amounts of acid, base, and/or buffering agent. Examples of suitable acids include, but are not limited to, hydrochloric acid, lactic acid, citric acid, acetic acid, and phosphoric acid. An example of a suitable base includes sodium hydroxide. Examples of buffering agents include acetic acid/acetate and bicarbonate/carbonate. If aqueous solubility is poor, intravenous emulsifying agents such as CREMOPHOR® EL (polyoxyethylated castor oil, CrEL) can be also be used.

Dofetilide can be formulated in aqueous or non-aqueous solutions, suspensions, or emulsions for intravenous administration at a concentration of 1-1000 μg/mL, including 1, 2, 5, 10, 15, 20, 25, 30, 35, 40, 45, 50, 55, 60, 65, 70, 75, 80, 90, 100, 110, 120, 130, 140, 150, 160, 170, 180, 190, 200, 220, 240, 260, 280, 300, 320, 340, 360, 380, 400, 420, 440, 460, 480, 500, 550, 600, 650, 700, 750, 800, 850, 900, 950, 1000 μg/mL, 10-100 μg/mL, 20-80 μg/mL, 30-70 μg/mL, or 40-60 μg/mL.

Compositions for oral administration of the anti-arrhythmics, if desired, include powders or granules, suspensions or solutions in water or non-aqueous media, capsules, sachets, or tablets. Thickeners, flavorings, diluents, emulsifiers, dispersing aids or binders may be desirable. Pharmaceutically acceptable carriers include fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries, flow-regulating agents and lubricants, such as silica, talc, stearic acid or salts thereof, such as magnesium stearate or calcium stearate, and/or polyethylene glycol can be added. In one implementation, dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate, are used. Slow dissolving polymers such as poly(bis(p-carboxyphenoxy)-propane:sebacic acid-CCP:SA) may also be used to generate wafers or beads that control or time the release of the composition. Dye stuffs or pigments may be added to the tablets or dragee coatings, for example, for identification or in order to characterize combinations of active compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules or nanoparticles which may optionally be mixed with fillers such as lactose, binders such as starches, and/or lubricants such as talc or magnesium stearate and, optionally, stabilizers. The active compound can be dissolved or suspended in suitable liquids, such as fatty oils, or liquid paraffin, optionally with stabilizers. LABRASOL®, a PEG derivative of medium chain fatty acid triglycerides of capric and caprylic acid, can be used as a pharmaceutical carrier and solubilizing agent to increase oral bioavailability if water solubility is poor.

Dofetilide is currently formulated and marketed as TIKOSYN® capsules containing the following inactive ingredients: microcrystalline cellulose, corn starch, colloidal silicon dioxide and magnesium stearate. TIKOSYN® is supplied for oral administration in three dosage strengths: 125 mcg (0.125 mg) orange and white capsules, 250 mcg (0.25 mg) peach capsules, and 500 mcg (0.5 mg) peach and white capsules.

Dofetilide has been experimentally infused intravenously in patients in pharmacokinetic and pharmacodynamic studies (see H. S. Rasmussen et al., Dofetilide, A Novel Class III Antiarrhythmic Agent, J Cardiovasc Pharmacol. 1992; 20 Suppl 2:S96-105 and M. Sedgwick et al., Pharmacokinetic and pharmacodynamic effects of UK-68,798, a new potential class III antiarrhythmic drug, Br. J. Clin. Pharmac. (1991), 31, 515-519 (“Sedgwick et al., 1991”) each incorporated by reference herein in its entirety). Sedgwick formulated dofetilide as a free base in a liquid vehicle containing 50 mg/ml mannitol and 4 mg/ml citric acid, titrated to pH 3.5 with sodium hydroxide. Sedgwick found dofetilide “easily soluble in aqueous solution”. Another investigator formulated dofetilide in 5% glucose as diluent (see M. F. Rousseau, Cardiac and Hemodynamic Effects of Intravenous Dofetilide in Patients With Heart Failure, Am J Cardiol 2001; 87:1250-1254). Various examples of IV formulations of dofetilide using vehicles containing one or more of 1,3-propanediol, HCl, acetic acid, sodium acetate, N,N-Dimethylacetamide, D-glucose and water, at a pH ranging from 4 to 7 have been described. (US Patent Application Publication No. 20190388371A1, incorporated by reference herein in its entirety). The dofetilide concentration was generally 50 μg/mL. Liposomal compositions for intravenous administration which include dofetilide have been described (see U.S. Pat. No. 9,682,041B2).

### Intravenous Administration

First order kinetics with a linear dose plasma concentration relationship, and a linear relationship between plasma concentration and effect on QT interval during IV administration of dofetilide have been described. (Sedgwick et al., 1991). Such kinetics allows titration of an appropriate anti-arrhythmic therapeutic plasma concentration and effect. The elimination half-life is approximately ten hours after intravenous or oral administration, and elimination is primarily renal (80 percent). Clearance has been estimated to be 0.35 L/hour/kg (Le Coz F, et al. Pharmacokinetic and pharmacodynamic modeling of the effects of oral and intravenous administrations of dofetilide on ventricular repolarization. Clin Pharmacol Ther 1995; 57:533).

According to embodiments, Aspect 1 is a method comprising: administering an anti-arrhythmic, such as dofetilide, to a patient, wherein the anti-arrhythmic or dofetilide is administered intravenously over a duration of at least 1 hour and in an amount effective for treating or preventing a cardiovascular condition of the patient.

Aspect 2 is a method, comprising: administering an anti-arrhythmic or dofetilide to a patient; wherein the anti-arrhythmic or dofetilide is administered: as a loading dose intravenously, optionally over a duration of up to 60 minutes, and in an amount ranging from 0.1 to 12 μg/kg bodyweight; and/or as a maintenance dose intravenously, optionally over a duration of at least 1 hour, and in an amount ranging from 0.1 to 10 μg/kg/hr, optionally alternatively or in addition wherein the amount of the IV loading dose is in the range of about ±50% of the IV maintenance dose.

Aspect 3 is the method of Aspect 1 or 2, wherein: the cardiovascular condition is selected from atrial fibrillation, atrial flutter, ventricular tachycardia, ventricular fibrillation, paroxysmal supraventricular tachycardia, heart failure, coronary artery disease, and pulmonary artery hypertension.

Aspect 4 is the method of any of Aspects 1-3, further comprising measuring a QT interval of the patient before, during, and/or after the administering, and selecting or adjusting the effective amount, the loading dose and/or the maintenance dose based on the QT interval.

Aspect 5 is the method of any of Aspects 1-4, further comprising measuring a creatinine clearance of the patient before the administering, and selecting or adjusting the effective amount, the loading dose and/or the maintenance dose based on the creatinine clearance.

Aspect 6 is a method of administering an anti-arrhythmic or dofetilide to a patient, comprising: (A) determining a creatinine clearance of the patient; (B) administering to the patient an IV loading dose of the anti-arrhythmic or dofetilide selected from an amount ranging from 0.1-12 μg/kg; (C) administering to the patient one or more IV maintenance dose of the anti-arrhythmic or dofetilide selected from an amount ranging from 0.1-10 μg/kg; and (D) optionally alternatively or in addition wherein the amount of the IV loading dose is in the range of about ±50% of the IV maintenance dose.

Aspect 7 is the method of any of Aspects 1-6, wherein the IV loading dose is 0.1 μg/kg, 0.5 μg/kg, 0.8 μg/kg, 1.5 μg/kg, 3.5 μg/kg, 4.5 μg/kg, 5.5 μg/kg, 6 μg/kg, 6.5 μg/kg, 7 μg/kg, 7.5 μg/kg, 8.5 μg/kg, 9 μg/kg, 9.5 μg/kg, 10.5 μg/kg, 11 μg/kg, or 11.5 μg/kg.

Aspect 8 is the method of any of Aspects 1-7, wherein the IV maintenance dose is 0.1 μg/kg, 0.5 μg/kg, 0.8 μg/kg, 1.5 μg/kg, 3.5 μg/kg, 4.5 μg/kg, 5.5 μg/kg, 6 μg/kg, 6.5 μg/kg, 7 μg/kg, 7.5 μg/kg, 8.5 μg/kg, 9 μg/kg, 9.5 μg/kg.

Aspect 9 is the method of any of Aspects 1-8, wherein the IV maintenance dose is lower than the IV loading dose.

Aspect 10 is the method of any of Aspects 1-8, wherein the IV maintenance dose is higher than the IV loading dose.

Aspect 11 is the method of any of Aspects 1-10, further comprising administering one or more oral dose of the anti-arrhythmic or dofetilide to the patient.

Aspect 12 is the method of any of Aspects 1-11, further comprising a time delay between completion of the administering of the IV loading dose and administering the one or more IV maintenance dose to the patient and/or administering the one or more oral dose to the patient.

Aspect 13 is the method of Aspect 12, wherein the time delay is less than 1 hour.

Aspect 14 is the method of Aspect 12, wherein the time delay is 1, 2, 3, 4, 5, or 6 hours.

Aspect 15 is the method of any of Aspects 1-8, wherein the IV maintenance dose is the same as the IV loading dose.

Aspect 16 is the method of Aspect 12, wherein the time delay is less than 30 minutes.

Aspect 17 is the method of any of Aspects 1-16, wherein the patient is being treated for atrial fibrillation and/or atrial flutter.

Aspect 18 is a method of administering an anti-arrhythmic or dofetilide to a patient, comprising: (A) determining a creatinine clearance of the patient; (B) administering to the patient an IV loading dose of the anti-arrhythmic or dofetilide selected from an amount ranging from 0.1-12 μg/kg; (C) administering to the patient (i) one or more IV maintenance dose of the anti-arrhythmic or dofetilide selected from an amount ranging from 0.1-10 μg/kg and/or (ii) one or more oral maintenance dose of the anti-arrhythmic or dofetilide selected from an amount ranging from 125 μg to 500 μg, such as 125 μg, 250 ag, or 500 ag; and (D) optionally alternatively or in addition wherein the amount of the IV loading dose is in the range of about ±50% of the oral dose and/or the IV maintenance dose.

Aspect 19 is the method of Aspect 18, wherein the creatinine clearance of the patient is >60 mL/min and the oral maintenance dose is 500 ag.

Aspect 20 is the method of Aspect 18, wherein the creatinine clearance of the patient is 40-60 mL/min and the oral maintenance dose is 250 ag.

Aspect 21 is the method of Aspect 18, wherein the creatinine clearance of the patient is 20-40 mL/min and the oral maintenance dose is 125 μg.

Aspect 22 is the method of Aspect 18, wherein the creatinine clearance of the patient is 20-60 mL/min and the IV loading dose is 3.5 μg/kg.

Aspect 23 is a method of initiating anti-arrhythmic or dofetilide treatment in a patient, comprising: (A) determining a creatinine clearance of the patient; (B) determining a QT interval of the patient; (C) administering to the patient an IV loading dose of an anti-arrhythmic or dofetilide selected from an amount ranging from 0.1-12 μg/kg; and (D) determining a second QT interval of the patient; (E) optionally administering one or more subsequent IV and/or oral doses of the anti-arrhythmic or dofetilide to the patient; (F) optionally alternatively or in addition wherein the amount of the IV loading dose is in the range of about ±50% of the subsequent IV dose and/or oral dose; and (G) optionally determining one or more subsequent QT interval of the patient between one or more of the subsequent IV and/or oral doses.

Aspect 24 is the method of Aspect 23, further comprising: determining a change in QT interval of the patient; and if the second QT interval has increased less than 15% over the first QT interval, then administering to the patient one or more IV maintenance dose selected from an amount ranging from 0.1-10 μg/kg and/or one or more oral maintenance dose selected from an amount of 125 μg, 250 μg, or 500 ag.

Aspect 25 is the method of any of Aspects 1-24, wherein the patient is experiencing or has been diagnosed with atrial fibrillation, atrial flutter, ventricular tachycardia, ventricular fibrillation, paroxysmal supraventricular tachycardia, heart failure, coronary artery disease, and/or pulmonary artery hypertension.

Aspect 26 is a method of initiating or escalating anti-arrhythmic or dofetilide treatment in a patient having atrial fibrillation or atrial flutter, comprising: (A) determining a creatinine clearance of the patient wherein the creatinine clearance of the patient is 20 to 60 mL/min; (B) determining a QT interval of the patient; (C) administering to the patient an IV loading dose of an anti-arrhythmic or dofetilide, wherein the IV loading dose is selected from an amount ranging from 0.1-1.6 μg/kg and wherein optionally the IV loading dose is administered over 5, 10, 15, 20, 30, 45, or 60 minutes, optionally by way of several IV doses; (D) determining a second QT interval of the patient; (E) administering one or more subsequent IV and/or oral doses of the anti-arrhythmic or dofetilide to the patient beginning immediately, from 1-6 hours, from 2-6 hours, or from 2-4 hours after completion of the administering of the IV loading dose; (F) optionally determining one or more subsequent QT interval of the patient between one or more of the subsequent IV or oral doses; (G) optionally administering one or more subsequent oral doses to the patient every 12 to 48 hours, wherein the oral dose administered is 125 μg for a patient with a creatinine clearance of 20 to <40 mL/min or 250 μg for a creatinine clearance of 40 to 60 mL/min; and (H) optionally alternatively or in addition wherein the amount of the IV loading dose is in the range of about ±50% of one or more of the subsequent IV or oral doses.

Aspect 27 is a method of initiating or escalating anti-arrhythmic or dofetilide treatment in a patient having atrial fibrillation (AF), atrial flutter (AFL) or both comprising: determining a creatinine clearance of the patient; intravenously administering an anti-arrhythmic or dofetilide according to a dosing regimen, wherein the dosing regimen is chosen from: (a) an IV loading dose of the anti-arrhythmic or dofetilide based on an oral target of 500 μg and one or more subsequent IV doses of the anti-arrhythmic or dofetilide, wherein the IV loading dose and the one or more subsequent IV doses are selected from an amount ranging from 1.6-3 μg/kg; (b) an IV loading dose of the anti-arrhythmic or dofetilide based on an oral target of 250 μg and one or more subsequent IV doses of the anti-arrhythmic or dofetilide, wherein the IV loading dose and the one or more subsequent IV doses are selected from an amount ranging from 0.8-1.6 μg/kg; or (c) an IV loading dose of the anti-arrhythmic or dofetilide based on an oral target of 125 μg and one or more subsequent IV doses of the anti-arrhythmic or dofetilide, wherein the IV loading dose and the one or more subsequent IV doses are selected from an amount ranging from 0.1-0.8 μg/kg; wherein the dosing regimen is chosen based on the creatinine clearance of the patient, such that dosing regimen (a) is chosen for a creatinine clearance of >60 mL/min, dosing regimen (b) is chosen for a creatinine clearance of 40 to 60 mL/min, and dosing regimen (c) is chosen for a creatinine clearance of 20 to <40 mL/min; wherein optionally the IV loading dose is administered over 10, 15, 20, 30, 45, or 60 minutes, optionally by way of several IV doses; wherein optionally one or more of the subsequent IV doses is administered by way of an infusion given over a time period of at least 1 hour; and optionally alternatively or in addition wherein the amount of the IV loading dose and/or one or more of the subsequent IV doses is in the range of about ±50% of the target oral dose.

Aspect 28 is a method of initiating or escalating an anti-arrhythmic or dofetilide treatment in a patient having atrial fibrillation or atrial flutter, comprising: (A) determining a creatinine clearance of the patient; (B) determining a QT interval of the patient; (C) administering to the patient an IV loading dose of an anti-arrhythmic or dofetilide, wherein the IV loading dose is chosen from an amount ranging from: i. 1.6-3 μg/kg, wherein the creatinine clearance of the patient is >60 mL/min; ii. 0.8-1.6 μg/kg, wherein the creatinine clearance of the patient is 40 to 60 mL/min; or iii. 0.1-0.8 μg/kg, wherein the creatinine clearance of the patient is 20 to <40 mL/min; (D) determining a second QT interval of the patient; (E) administering one or more oral doses of the anti-arrhythmic or dofetilide to the patient beginning during the administering of the IV loading dose or immediately, 1-6 hours, or 2-6 hours after completion of the administering of the IV loading dose; (F) optionally determining one or more subsequent QT interval of the patient between one or more of the subsequent oral doses; and (G) optionally alternatively or in addition wherein the amount of the IV loading dose is in the range of about ±50% of one or more of the oral doses.

Aspect 29 is a method of initiating or escalating an anti-arrhythmic or dofetilide treatment in a patient having atrial fibrillation or atrial flutter, comprising: (A) determining a QT interval of a patient with atrial fibrillation or atrial flutter; (B) determining the patient has a creatinine clearance in the range of 20 to 60 mL/min; (C) administering to the patient an IV loading dose of an anti-arrhythmic or dofetilide selected from an amount ranging from 0.1-1.6 μg/kg and optionally administered over a period of from 5 min. to 1 hour; (D) determining a second QT interval of the patient; (E) administering to the patient one or more subsequent IV or oral doses of the anti-arrhythmic or dofetilide beginning immediately, 1-6 hrs, 2-6 hrs, or 2-4 hrs after completion of the IV loading dose; (F) wherein the subsequent IV doses are selected from an amount ranging from 0.1-1.6 μg/kg and optionally administered over a period of from 5 min. to 1 hour; (G) wherein the subsequent oral doses are selected from an amount ranging from 125-250 μg, optionally administered every 12-48 hours; (H) optionally determining one or more subsequent QT interval of the patient between one or more of the subsequent IV and/or oral doses; and (I) optionally alternatively or in addition wherein the amount of the IV loading dose is in the range of about ±50% of one or more of the subsequent IV or oral doses.

Aspect 30 is a method of initiating or escalating an anti-arrhythmic or dofetilide treatment in a patient having atrial fibrillation or atrial flutter, comprising: (A) determining a creatinine clearance of the patient; (B) determining a QT interval of the patient; (C) administering to the patient an IV loading dose of an anti-arrhythmic or dofetilide selected from an amount ranging from: i. 1.6-3 μg/kg, wherein the creatinine clearance of the patient is >60 mL/min; ii. 0.8-1.6 μg/kg, wherein the creatinine clearance of the patient is 40 to 60 mL/min; or iii. 0.1-0.8 μg/kg, wherein the creatinine clearance of the patient is 20 to <40 mL/min; and (D) administering a first oral dose of the anti-arrhythmic or dofetilide to the patient before, during, or upon completion of the IV loading dose, wherein the oral dose is chosen from: i. 500 μg, wherein the creatinine clearance of the patient is >60 mL/min; ii. 250 μg, wherein the creatinine clearance of the patient is 40 to 60 mL/min; or iii. 125 μg, wherein the creatinine clearance of the patient is 20 to <40 mL/min; (E) determining a second QT interval of the patient; (F) administering one or more subsequent oral dose of the anti-arrhythmic or dofetilide to the patient every 12-48 hours; (G) optionally determining one or more subsequent QT interval of the patient between one or more of the subsequent oral doses; (H) optionally alternatively or in addition wherein the amount of the IV loading dose is in the range of about ±50% of the first oral dose and/or one or more of the subsequent oral doses.

Aspect 31 is a method of initiating or escalating anti-arrhythmic or dofetilide treatment in a patient having atrial fibrillation or atrial flutter, comprising: (A) determining a creatinine clearance of the patient; (B) determining a QT interval of the patient; (C) administering to the patient an IV loading dose of an anti-arrhythmic or dofetilide, wherein the IV loading dose is selected from an amount ranging from 0.1 to 3 μg/kg; and (D) administering a first oral dose of the anti-arrhythmic or dofetilide to the patient before, during, or upon completion of the IV loading dose, wherein the oral dose is chosen from: i. 500 μg, wherein the creatinine clearance of the patient is >60 mL/min; ii. 250 μg, wherein the creatinine clearance of the patient is 40 to 60 mL/min; or iii. 125 μg, wherein the creatinine clearance of the patient is 20 to <40 mL/min; (E) determining a second QT interval of the patient; (F) administering one or more subsequent oral dose to the patient every 12-48 hours; (G) optionally determining one or more subsequent QT interval of the patient between one or more of the subsequent oral doses; (H) optionally alternatively or in addition wherein the amount of the IV loading dose is in the range of about ±50% of the first oral dose and/or one or more of the subsequent oral doses.

Aspect 32 is a method of initiating or escalating anti-arrhythmic or dofetilide treatment in a patient having atrial fibrillation or atrial flutter, comprising: (A) determining a creatinine clearance of the patient; (B) determining a QT interval of the patient; (C) administering to the patient an IV loading dose of an anti-arrhythmic or dofetilide, wherein the IV loading dose is selected from an amount ranging from: i. 1.6-3 μg/kg, wherein the creatinine clearance of the patient is >60 mL/min; ii. 0.8-1.6 μg/kg, wherein the creatinine clearance of the patient is 40 to 60 mL/min; or iii. 0.1-0.8 μg/kg, wherein the creatinine clearance of the patient is 20 to <40 mL/min; and (D) administering a first oral dose of the anti-arrhythmic or dofetilide to the patient before, during, or upon completion of the IV loading dose, wherein the first oral dose is selected from 125 μg, 250 μg, or 500 μg; (E) determining a second QT interval of the patient; (F) administering one or more subsequent oral dose of the anti-arrhythmic or dofetilide to the patient every 12-48 hours; and (G) optionally determining one or more subsequent QT interval of the patient between one or more of the subsequent oral doses; (H) optionally alternatively or in addition wherein the amount of the IV loading dose is in the range of about ±50% of the first oral dose and/or one or more of the subsequent oral doses.

Aspect 33 is a method of initiating or escalating an anti-arrhythmic or dofetilide treatment in a patient having atrial fibrillation or atrial flutter, comprising: (A) determining a creatinine clearance of the patient; (B) determining a QT interval of the patient; (C) administering to the patient an IV loading dose of the anti-arrhythmic or dofetilide, wherein the IV loading dose is selected from an amount ranging from 0.1 to 3 μg/kg; (D) administering a first oral dose of the anti-arrhythmic or dofetilide to the patient before, during, or upon completion of the IV loading dose, wherein the first oral dose is chosen from 125 μg, 250 μg, or 500 μg; (E) determining a second QT interval of the patient; (F) administering one or more subsequent oral dose of the anti-arrhythmic or dofetilide to the patient every 12-48 hours, wherein one or more of the subsequent oral doses is a higher dose or a lower dose than the first oral dose; and (G) optionally determining one or more subsequent QT interval of the patient between one or more of the subsequent oral doses; (H) optionally alternatively or in addition wherein the amount of the IV loading dose is in the range of about ±50% of the first oral dose and/or one or more of the subsequent oral doses.

Aspect 34 is a method of initiating or escalating anti-arrhythmic or dofetilide treatment in a patient having atrial fibrillation or atrial flutter, comprising: (A) determining a creatinine clearance of the patient; (B) determining a first QT interval of the patient; (C) administering to the patient an IV loading dose of the anti-arrhythmic or dofetilide, wherein the IV loading dose is selected from an amount ranging from 0.1 to 3 μg/kg; (D) administering a first oral dose of the anti-arrhythmic or dofetilide to the patient before, during, or upon completion of the IV loading dose, wherein the first oral dose is chosen from 125 μg, 250 μg, or 500 μg; (E) determining a second QT interval of the patient that is longer than the first QT interval of the patient; (F) administering one or more subsequent oral dose of the anti-arrhythmic or dofetilide to the patient every 12-48 hours, wherein the one or more subsequent oral dose is a higher dose than the first oral dose to further increase the second QT interval; and (G) optionally determining one or more subsequent QT interval of the patient between one or more of the subsequent oral doses; (H) optionally alternatively or in addition wherein the amount of the IV loading dose is in the range of about ±50% of the first oral dose and/or one or more of the subsequent oral doses.

Aspect 35 is a method of initiating or escalating anti-arrhythmic or dofetilide treatment in a patient having atrial fibrillation or atrial flutter, comprising: (A) determining a creatinine clearance of the patient; (B) determining a first QT interval of the patient; (C) administering to the patient an IV loading dose of the anti-arrhythmic or dofetilide, wherein the IV loading dose is selected from an amount ranging from 0.1 to 3 μg/kg; (D) administering a first oral dose of the anti-arrhythmic or dofetilide to the patient before, during, or upon completion of the IV loading dose, wherein the first oral dose is chosen from 125 μg, 250 μg, or 500 μg; (E) determining a second QT interval of the patient that is longer than the first QT interval of the patient; (F) administering one or more subsequent oral dose of the anti-arrhythmic or dofetilide to the patient every 12-48 hours, wherein the one or more subsequent oral dose is a lower dose than the first oral dose to reduce the QT interval; (G) optionally determining one or more subsequent QT interval of the patient between one or more of the subsequent oral doses; and (H) optionally alternatively or in addition wherein the amount of the IV loading dose is in the range of about ±50% of the first oral dose and/or one or more of the subsequent oral doses.

Aspect 36 is the method of any of Aspects 1-35, wherein the anti-arrhythmic or dofetilide, and/or the IV loading dose, and/or one or more subsequent IV doses, and/or one or more of the IV maintenance doses is administered over 5, 10, 15, 20, 30, 45, or 60 minutes, is administered for up to 60 minutes, is administered over a time period of at least 1 hour, is administered over 0.5, 1, 2, 3, 4, 5, or 6 hours, or is administered for a duration of at least 1 hour, and/or optionally is administered by way of several IV doses.

Aspect 37 is the method of any of Aspects 1-36, wherein the anti-arrhythmic or dofetilide, and/or the IV loading dose, and/or one or more subsequent IV doses, and/or one or more of the IV maintenance doses is administered to a patient who is unable to take (NPO) oral anti-arrhythmics or dofetilide.

Aspect 38 is the method of any of Aspects 1-37, wherein the anti-arrhythmic is one or more of the following drugs: sotalol, amiodarone, ibutilide, dronedarone, procainamide, flecainide, or propafenone, optionally wherein the dosing is adjusted for pharmacokinetics of the drug being administered.

Aspect 39 is a method, comprising: administering an antiarrhythmic, such as dofetilide, to a subject; wherein the subject is a subject with atrial fibrillation cardioverted to normal sinus rhythm, without ventricular arrhythmias or various forms of blocks, and with normal kidney function; and wherein the antiarrhythmic or dofetilide is administered intravenously as a loading dose over a duration of at least 1 hour and in an amount effective for maintenance of normal sinus rhythm.

Aspect 40 is the method of Aspect 39, wherein the amount of the loading dose for dofetilide ranges from 0.1 to 12 μg/kg bodyweight of the subject.

Aspect 41 is the method of Aspect 39 or 40, wherein the amount of the intravenous loading dose for dofetilide is less than 1.8 μg/kg or greater than 3 μg/kg.

Aspect 42 is the method of any of Aspects 39-41, further comprising administering at least one maintenance dose of intravenous and/or oral dofetilide, and optionally alternatively or in addition wherein the amount of the loading dose is in the range of about ±50% of the IV or oral maintenance dose.

Aspect 43 is the method of Aspect 42, wherein the oral dofetilide maintenance dose is in an amount ranging from 125 to 500 μg.

Aspect 44 is the method of Aspect 42 or 43, wherein the intravenous dofetilide maintenance dose is in an amount ranging from 0.1 to 12 μg/kg bodyweight of the subject, such as up to or less than 1.8 μg/kg, such as at least or greater than 3 μg/kg, such as from above 0 up to 1.8 μg/kg, such as from above 0 up to 3 μg/kg, or from 1.8 μg/kg to 3 μg/kg.

Aspect 45 is the method of any of Aspects 42-44, wherein the maintenance oral or IV dofetilide dose(s) is/are administered at an interval of 12-72 hrs.

Aspect 46 is the method of any of Aspects 42-45, wherein the maintenance oral or IV dofetilide dose(s) is/are administered immediately following the IV loading dose, or 0.5 hours after, up to 1 hour after, from 1-6 hours after, from 2-6 hours after, from 2-3 hours after, from 2-4 hours after, from 2-5 hours after, or from 2-12 hours after the IV loading dose.

Aspect 47 is the method of any of Aspects 42-46, wherein the IV maintenance dose is administered for a duration ranging from up to or including any one or more of 5 hours, 4 hours, 3 hours, 2 hours, 1 hour, 45 min., 30 min., 15 min., 10 min., or 5 min.

Aspect 48 is the method of any of Aspects 39-47, wherein the subject is a subject who is unable (NPO) to take oral dofetilide.

Aspect 49 is a method, comprising: administering an antiarrhythmic, such as dofetilide, to a subject; wherein the subject is a subject with atrial fibrillation cardioverted to normal sinus rhythm, without ventricular arrhythmias or various forms of blocks, and with reduced kidney function; and wherein the antiarrhythmic or dofetilide is administered intravenously as a loading dose over a duration of at least 1 hour and in an amount effective for maintenance of normal sinus rhythm.

Aspect 50 is the method of Aspect 49, wherein the subject has a creatinine clearance of 20 mL/min to 40 mL/min.

Aspect 51 is the method of Aspect 49, wherein the subject has a creatinine clearance of 40 mL/min to 60 mL/min.

Aspect 52 is the method of Aspect 49, wherein the subject has a creatinine clearance of 60 mL/min to 90 mL/min.

Aspect 53 is the method of any of Aspects 49-52, wherein the amount of the loading dose for dofetilide ranges from 0.1 to 12 μg/kg bodyweight of the subject.

Aspect 54 is the method of any of Aspects 49-52, wherein the amount of the intravenous loading dose for dofetilide is less than 1.8 μg/kg or greater than 3 μg/kg.

Aspect 55 is the method of any of Aspects 49-54, further comprising administering at least one maintenance dose of intravenous and/or oral dofetilide, and optionally alternatively or in addition wherein the amount of the loading dose is in the range of about ±50% of one or more of the maintenances doses.

Aspect 56 is the method of Aspect 55, wherein the maintenance dose is one or more oral dofetilide maintenance dose and is in an amount ranging from 125 to 500 μg, and optionally alternatively or in addition wherein the amount of the loading dose is in the range of about ±50% of one or more of the oral dofetilide maintenances doses.

Aspect 57 is the method of Aspect 55 or 56, wherein the intravenous dofetilide maintenance dose is in an amount ranging from 0.1 to 12 μg/kg bodyweight of the subject, such as up to or less than 1.8 μg/kg, such as at least or greater than 3 μg/kg, such as from above 0 up to 1.8 μg/kg, such as from above 0 up to 3 μg/kg, or from 1.8 μg/kg to 3 μg/kg.

Aspect 58 is the method of any of Aspects 55-57, wherein the maintenance oral or IV dofetilide dose(s) is/are administered at an interval of 12-72 hrs.

Aspect 59 is the method of any of Aspects 55-58, wherein the maintenance oral or IV dofetilide dose(s) is/are administered immediately following the IV loading dose, or 0.5 hours after, up to 1 hour after, from 1-6 hours after, from 2-6 hours after, from 2-3 hours after, from 2-4 hours after, from 2-5 hours after, or from 2-12 hours after the IV loading dose.

Aspect 60 is the method of any of Aspects 55-59, wherein the IV maintenance dose is administered for a duration ranging from up to or including any one or more of 5 hours, 4 hours, 3 hours, 2 hours, 1 hour, 45 min., 30 min., 15 min., 10 min., or 5 min.

Aspect 61 is the method of any of Aspects 49-60, wherein the subject is a subject who is unable (NPO) to take oral dofetilide.

Aspect 62 is a method of initiating dofetilide treatment, comprising: a. determining a QTc of a patient; b. administering a loading dose of dofetilide intravenously to the patient; c. determining a second QTc of the patient; d. administering a first maintenance dose of dofetilide orally or intravenously to the patient; and e. optionally administering one or more subsequent maintenance dose of dofetilide orally or intravenously to the patient.

Aspect 63 is the method of any of Aspects 1-62, wherein the loading dose is divided into multiple intravenous doses optionally administered to the patient with a delay between each dose.

Aspect 64 is the method of any of Aspects 1-63, wherein the loading dose is selected from an amount in the range of about ±50% of the amount of the oral dose.

Aspect 65 is the method of any of Aspects 1-64, wherein the loading dose is selected from an amount in the range of about ±25% of the amount of the oral dose.

Aspect 66 is the method of any of Aspects 1-65, wherein the loading dose is selected from an amount in the range of about ±50% of the amount of the maintenance dose.

Aspect 67 is the method of any of Aspects 1-66, wherein the loading dose is selected from an amount in the range of about ±25% of the amount of the maintenance dose.

According to some more specific implementations, the anti-arrhythmic or dofetilide can be administered intravenously as a loading dose in the range of above 0 μg/kg up to 8 μg/kg, including 0.1, 0.2, 0.3, 0.4, 0.5, 0.6, 0.7, 0.8, 0.9, 1.0, 1.2, 1.4, 1.5, 1.6, 1.8, 2.0, 2.2, 2.4, 2.5, 2.6, 2.8, 3.0, 3.2, 3.4, 3.5, 3.6, 3.8, 4.0, 4.2, 4.4, 4.5, 4.6, 4.8, 5.0, 5.2, 5.4, 5.5, 5.6, 5.8, 6.0, 6.2, 6.4, 6.5, 6.6, 6.8, 7.0, 7.2, 7.4, 7.5, 7.6, 7.8, 8.0, μg/kg, or in any range with any of these values as lower or upper values, such as 1.5 to 4.4 μg/kg, 3.2 to 6.8 μg/kg, 2.6 to 5.4 μg/kg, 2.0 to 6.0 μg/kg, 1.8 to 4.8 μg/kg, and so on. Depending on the particular anti-arrhythmic selected (such as for sotalol, amiodarone, ibutilide, dronedarone, procainamide, flecainide, or propafenone), the dosing can be adjusted up or down to be commensurate with the pharmacokinetics of the drug being administered. Higher loading doses, such as those exceeding 8.0 μg/kg, such as 8.2 to 12.0 μg/kg, including 8.2, 8.4, 8.5, 8.6, 8.8, 9.0, 9.2, 9.4, 9.5, 9.6, 9.8, 10.0, 10.2, 10.4, 10.5, 10.6, 10.8, 11.0, 11.2, 11.4, 11.5, 11.6, 11.8, and 12.0 μg/kg, or in any range with any of these values as lower and upper values, such as 8.2 to 10 μg/kg may be possible, such as, if administered over longer durations. The loading dose can be slowly titrated upward until restoration of normal sinus rhythm as measured by electrocardiogram (ECG), or ceased or adjusted downward if ventricular arrhythmias, ventricular tachycardia, or other side effects of dofetilide such as dizziness are observed. The loading dose can be adjusted for patient QTc as described below. The loading dose can be adjusted for patient creatinine clearance as described below. The loading dose can be administered over 0.5 to 60 minutes or longer, including for 0.5, 1, 2, 3, 4, 5, 6, 7, 8, 9, 10, 11, 12, 13, 14, 15, 20, 25, 30, 35, 40, 45, 50, 55, or 60 minutes, or within any range with any of these values as lower and upper values, such as 5 to 20 minutes, 10 to 40 minutes, 15 to 30 minutes, 20 to 45 minutes, 30 to 60 minutes, 35 to 50 minutes, 35 to 60 minutes, 40 to 60 minutes, 45 to 60 minutes, and so on. Similar to the changes made in dose for a particular anti-arrhythmic being used, depending on the particular anti-arrhythmic selected (such as for sotalol, amiodarone, ibutilide, dronedarone, procainamide, flecainide, or propafenone), the duration of the IV administration can be adjusted up or down to be commensurate with the pharmacokinetics of the drug being administered. The loading dose can be intravenously infused into a central or peripheral vein of the patient. The flow rate of the intravenous dose can be adjusted based on the concentration of the anti-arrhythmic or dofetilide in the intravenous formulation, the desired duration of administration, and the body weight of the patient to achieve a specific loading dose in μg/kg or mg/kg, if appropriate for the particular anti-arrhythmic being used. Thus, longer administration durations of a specific dose (X μg/kg or mg/kg) will typically have slower flow rates compared to shorter administration durations to achieve the same dose. In embodiments, the intravenous loading dose amount is selected from an amount in the range of about ±50% of an oral dose or maintenance dose, such as an amount of up to about ±5%, 10%, 15%, 20%, 25%, ±30%, 35%, 40%, or ±45%, or any range in between.

According to some implementations, after the loading dose, the anti-arrhythmic or dofetilide can be infused intravenously as a maintenance dose rate in the range of above 0 μg/kg/hr up to 10 μg/kg/hr, including 0.1, 0.2, 0.3, 0.4, 0.5, 0.6, 0.7, 0.8, 0.9, 1.0, 1.2, 1.3, 1.4, 1.5, 1.6, 1.8, 1.9, 2.0, 2.2, 2.4, 2.5, 2.6, 2.7, 2.8, 3.0, 3.1, 3.2, 3.3, 3.4, 3.5, 3.6, 3.8, 4.0, 4.2, 4.3, 4.4, 4.5, 4.6, 4.7, 4.8, 5.0, 5.1, 5.2, 5.4, 5.5, 5.6, 5.7, 5.8, 6.0, 6.1, 6.2, 6.4, 6.5, 6.6, 6.8, 6.9, 7.0, 7.2, 7.4, 7.5, 7.6, 7.7, 7.8, 7.9, 8.0, 8.1, 8.2, 8.3, 8.4, 8.5, 8.6, 8.8, 8.9, 9.0, 9.2, 9.4, 9.5, 9.6, 9.7, 9.8, 9.9, 10.0 μg/kg/hr, or in any range with any of these values as lower or upper values, such as 1.2 to 2.8 μg/kg/hr, 2.6 to 7.4 μg/kg/hr, 3.4 to 5.8 μg/kg/hr, 4.2 to 8.6 μg/kg/hr, 1.0 to 3.0 μg/kg/hr, 2.8 to 3.6 μg/kg/hr, 1.0 to 4 μg/kg/hr, 1.2 to 4 μg/kg/hr, 1.4 to 4 μg/kg/hr, 1.6 to 4 μg/kg/hr, 1.8 to 4 μg/kg/hr, 2.0 to 4 μg/kg/hr, 2.2 to 4 μg/kg/hr, 2.4 to 4 μg/kg/hr, 2.6 to 4 μg/kg/hr, 2.7 to 4 μg/kg/hr, 2.8 to 4 μg/kg/hr, 1.0 to 5 μg/kg/hr, 1.2 to 5 μg/kg/hr, 1.4 to 5 μg/kg/hr, 1.6 to g/kg/hr, 1.8 to 5 μg/kg/hr, 2.0 to 5 μg/kg/hr, 2.2 to 5 μg/kg/hr, 2.4 to 5 μg/kg/hr, 2.6 to g/kg/hr, 2.7 to 5 μg/kg/hr, 2.8 to 5 μg/kg/hr, 3.0 to 5 μg/kg/hr, 3.2 to 5 μg/kg/hr, 3.4 to g/kg/hr, 3.6 to 5 μg/kg/hr, 1.0 to 6 μg/kg/hr, 1.2 to 6 μg/kg/hr, 1.4 to 6 μg/kg/hr, 1.6 to 6 μg/kg/hr, 1.8 to 6 μg/kg/hr, 2.0 to 6 μg/kg/hr, 2.2 to 6 μg/kg/hr, 2.4 to 6 μg/kg/hr, 2.6 to 6 μg/kg/hr, 2.7 to 6 μg/kg/hr, 2.8 to 6 μg/kg/hr, 3.0 to 6 μg/kg/hr, 3.2 to 6 μg/kg/hr, 3.2 to 6 μg/kg/hr, 3.4 to 6 μg/kg/hr, 3.6 to 6 μg/kg/hr, 3.8 to 6 μg/kg/hr, 4.0 to 6 μg/kg/hr, 1.0 to 8 μg/kg/hr, 1.2 to 8 μg/kg/hr, 1.4 to 8 μg/kg/hr, 1.6 to 8 μg/kg/hr, 1.8 to 8 μg/kg/hr, 2.0 to 8 μg/kg/hr, 2.2 to 8 μg/kg/hr, 2.4 to 8 μg/kg/hr, 2.6 to 8 μg/kg/hr, 2.7 to 8 μg/kg/hr, 2.8 to 8 μg/kg/hr, 3.0 to 8 μg/kg/hr, 3.2 to 8 μg/kg/hr, 3.4 to 8 μg/kg/hr, 3.6 to 8 μg/kg/hr, 3.8 to 8 μg/kg/hr, 4.0 to 8 μg/kg/hr, 4.2 to 8 μg/kg/hr, 4.4 to 8 μg/kg/hr, 4.6 to 8 μg/kg/hr, 4.8 to 8 μg/kg/hr, 5.0 to 8 μg/kg/hr, 1.0 to 10 μg/kg/hr, 1.2 to 10 μg/kg/hr, 1.4 to 10 μg/kg/hr, 1.6 to g/kg/hr, 1.8 to 10 μg/kg/hr, 2.0 to 10 μg/kg/hr, 2.2 to 10 μg/kg/hr, 2.4 to 10 μg/kg/hr, 2.6 to g/kg/hr, 2.7 to 10 μg/kg/hr, 2.8 to 10 μg/kg/hr, 3.0 to 10 μg/kg/hr, 3.2 to 10 μg/kg/hr, 3.4 to g/kg/hr, 3.6 to 10 μg/kg/hr, 3.8 to 10 μg/kg/hr, 4.0 to 10 μg/kg/hr, 4.2 to 10 μg/kg/hr, 4.4 to g/kg/hr, 4.6 to 10 μg/kg/hr, 4.8 to 10 μg/kg/hr, and 5.0 to 10 μg/kg/hr 5.2 to 10 μg/kg/hr, 5.4 to g/kg/hr, 5.6 to 10 μg/kg/hr, 5.8 to 10 μg/kg/hr, 6.0 to 10 μg/kg/hr, and so on. Depending on the particular anti-arrhythmic selected (such as for sotalol, amiodarone, ibutilide, dronedarone, procainamide, flecainide, or propafenone), the IV maintenance dose can be adjusted up or down to be commensurate with the pharmacokinetics of the drug being administered. In embodiments, the maintenance dose is initiated at a higher rate and then titrated down. In other embodiments, the maintenance dose is initiated at a lower rate and then increased.

The maintenance dose (IV or oral) can be administered over a duration of 30 minutes to several hours to several days to several months to several years, including 0.5, 1.0, 1.5, 2.0, 2.5, 3.0, 3.5, 4.0, 4.5, 5.0, 5.5, 6.0, 6.5, 7.0, 7.5, 8.0, 9, 10, 11, 12, 13, 14, 15, 16, 17, 18, 19, 20, 21, 22, 23, 24, 36, 48, 72 hours, including 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 days, or including 1, 2, 3, 4, 5, 6, 7, 8, 9, 10, 11, 12 months, or including 1, 2, 3, 4, 5, 8, or 10 years, or in any range with any of these values as lower and upper values, such as 0.5 to 1.5 hours, 1 to 2 hours, 1 to 3 hours, 1 to 4 hours, 1 to 5 hours, 1 to 6 hours, 1 to 10 hours, 1 to 18 hours, 1 to 24 hours, 1 to 36 hours, 1 to 48 hours, 1 to 72 hours, 1 to 168 hours, 2 to 3 hours, 2 to 4 hours, 2 to 5 hours, 2 to 6 hours, 2 to 8 hours, 2 to 10 hours, 2 to 18 hours, 2 to 24 hours, 2 to 36 hours, 2 to 48 hours, 2 to 72 hours, 2 to 168 hours, 3 to 4 hours, 3 to 5 hours, 3 to 6 hours, 3 to 10 hours, 3 to 18 hours, 3 to 24 hours, 3 to 36 hours, 3 to 48 hours, 3 to 72 hours, 3 to 168 hours, 4 to 8 hours, 4 to 10 hours, 4 to 18 hours, 4 to 24 hours, 4 to 36 hours, 4 to 48 hours, 4 to 72 hours, 4 to 168 hours, 6 to 8 hours, 6 to 10 hours, 6 to 18 hours, 6 to 24 hours, 6 to 36 hours, 6 to 48 hours, 6 to 72 hours, 6 to 168 hours, 8 to 10 hours, 8 to 18 hours, 8 to 24 hours, 8 to 36 hours, 8 to 48 hours, 8 to 72 hours, 8 to 168 hours, 12 to 24 hours, 12 to 72 hours, 12 to 168 hours, 24 to 72 hours, 1 to 7 days, 1 to 10 days, 1 to 14 days, 7 to 30 days, 1 to 3 months, 2 to 6 months, 1 to 5 years, and so on.

The maintenance dose can be initiated immediately after completion of the loading dose, or can be initiated after a delay of 0.5 hours to 6 hours, such as a delay of from 1, 1.5, 2, 2.5, 3, 3.5, 4, 4.5, 5, or 5.5 hours after completion of the loading dose.

The maintenance dose can be intravenously infused into a central or peripheral vein of the patient. In other embodiments, the maintenance dose can be given orally. In an embodiment, the maintenance dose is initiated as an IV infusion, then transitioned to oral administration. The patient can be monitored by electrocardiogram (ECG) for normal sinus rhythm and/or ventricular arrhythmias or ventricular tachycardia and the maintenance dose rate adjusted upward until normal sinus rhythm is reached or adjusted downward if ventricular arrhythmias, ventricular tachycardia, or other side effects typically associated with anti-arrhythmic or dofetilide administration, such as dizziness occur. The maintenance dose can be adjusted for patient QTc as described below. The maintenance dose rate can be adjusted for patient creatinine clearance as described below. The flow rate of the intravenous dose can be adjusted based on the concentration of dofetilide in the intravenous formulation and the body weight of the patient to achieve a specific maintenance dose rate in μg/kg/hr. The maintenance dose rate can be targeted to reach a particular therapeutic, steady-state plasma concentration based on established pharmacokinetic parameters of dofetilide such as half-life and clearance. As can be appreciated, the cumulative maintenance dose is a product of the rate of intravenous infusion in μg/kg/hr multiplied by the infusion duration in hours.

### QT Interval Monitoring

Proper dosing for any of dofetilide, sotalol, amiodarone, ibutilide, dronedarone, procainamide, flecainide, or propafenone can be ascertained by monitoring the QT interval, or the heart rate corrected QT (the QTc), during or after IV infusion of the loading and/or maintenance dose(s), and thus avoid the development of life-threatening ventricular tachycardias such as Torsades de Pointes. Patients can be monitored by electrocardiogram (ECG) and a baseline QTc can be measured. A baseline QTc exceeding a certain threshold, such as 440 msec, may exclude some patients from dofetilide treatment (see US Patent Application Publication No. 20190388371A1). For patients that qualify for treatment, the QTc can be monitored during infusion of the loading and/or maintenance dose, and such dose can be adjusted downward or ceased if the patient's QTc becomes too high, either over a QTc threshold or a change exceeding a patient's baseline QTc. In one implementation, the QTc threshold is 500 msec. In various implementations, the change exceeding baseline QTc can be 5, 10, 15, or 20% over baseline QTc. The QTc can be measured at regular intervals, such as every 10, 15, 20, 30, 45, or 60 minutes during treatment. In some situations, such as if the patient's heart rate is less than 60 bpm, the uncorrected QT interval can be used. Remote ECG monitoring, such as Holter monitoring or event monitoring, may be indicated for long term monitoring of QT or QTc.

### Creatinine Clearance

Because elimination of dofetilide is primarily renal, renal impairment can potentially lead to toxic serum concentrations. As such, it is desirable to measure patient creatinine clearance prior to initiating a dofetilide dosing regimen in order to gauge kidney function. Serum samples can be taken from the patient prior to treatment with dofetilide and serum creatinine measured according the following formulas:

$$\begin{matrix}Creatinine clearance ( male ) = [ ( 140 - Age ) \times Body Wt ( kg ) ] / [ 72 \times serum creatinine ( mg / dL ) ] \\ Creatinine clearance ( female ) = Creatine clearance ( male ) \times 0.85\end{matrix}$$

The loading dose and maintenance dose can be adjusted according to creatinine clearance as provided in Table I.

TABLE I

| Creatinine | | |
|---|---|---|
| Clearance | | |
| (mL/min) | Loading Dose | Maintenance Dose |
| >60 | High | High |
| 40-60 | Medium | Medium |
| 20-40 | Low | Low |
| <20 | Dofetilide not | Dofetilide not |
| | indicated | indicated |

### Indications

Dofetilide administered intravenously in any of the above loading and/or maintenance doses and durations can be used to treat or prevent such conditions as atrial fibrillation or flutter (sustained or intermittent), paroxysmal atrial fibrillation, ventricular tachycardia, ventricular fibrillation, paroxysmal supraventricular tachycardia, heart failure, coronary artery disease, pulmonary artery hypertension, and the like, and for situations where the patient is unable to take an anti-arrhythmic by mouth (NPO). Other anti-arrhythmics can be substituted for dofetilide, including other Class III anti-arrhythmics such as sotalol, amiodarone, ibutilide, and dronedarone or Class I anti-arrhythmics such as procainamide, flecainide, and propafenone. Adjustments to the dosing for a particular substitute can be made by one of ordinary skill in the art based on dosages appropriate for a particular drug. For example, similar dosages can be used for ibutilide, while for amiodarone, dronedarone, propafenone, procainamide, flecainide and sotalol, the dosing would be on the order of mg/kg instead of the μg/kg indicated for dofetilide. For some drugs the oral maintenance dose may be lower or higher than a corresponding dose for dofetilide and/or may be administered more frequently or less frequently than dofetilide. The specific intravenous loading dose and intravenous maintenance dose rate protocols would be selected from those described herein based upon the patient's condition, baseline and in-treatment QTc, as well as the patient's creatinine clearance. Other factors affecting selection of the loading and maintenance dose include patient body weight. In cases where there is no intravenous maintenance dose rate protocol (i.e. “none”), an oral maintenance dose of dofetilide can be administered to the patient according to available oral dosages (125 μg, 250 μg, 500 μg, typically given twice daily), QTc, creatinine clearance, body weight, and other factors, as described by U.S. Patent Application Publication No. 20190388371A1). Situations where IV loading and/or maintenance doses are appropriate include those in which the patient cannot take oral administration (NPO), or for gastrointestinal conditions resulting in poor absorption, recovery from GI surgery, and/or intensive care.

Dofetilide administration according to embodiments of the invention is administered to patients naïve to dofetilide treatment and/or to patients currently receiving dofetilide therapy and/or to patients having previously received dofetilide.

In embodiments, the dofetilide dose (including the loading dose and/or maintenance dose, whether IV or oral) is determined by patient characteristics including body weight, sex, and/or creatinine clearance.

In embodiments, antiarrhythmics, such as dofetilide, can be administered to subjects/patients hospitalized with atrial fibrillation cardioverted to normal sinus rhythm, typically without ventricular arrhythmias or various forms of blocks, and typically in patients with normal kidney function. In other embodiments, the patients have reduced kidney function or can have ventricular arrhythmias, blocks or other cardiovascular conditions/diseases.

Atrial fibrillation (AF) is a heart rhythm disorder caused by a degeneration of the electrical impulses of the atria resulting in a rapid, chaotic rhythm (also called an arrhythmia). This arrhythmia is a direct result of disordered impulses across the atrioventricular (AV) node, to the lower cardiac chambers (ventricles). The arrhythmia causes ineffectual atrial contractions, which affect cardiac output and leads to vulnerability to thrombus formation that can result in strokes and related cardiovascular accidents. (See https://www.clevelandclinicmeded.com/medicalpubs/diseasemanagement/cardiology/atrial-fibrillation/, which is hereby incorporated by reference herein in its entirety.)

Unstable patients presenting with AF, such as patients with chest pain, pulmonary edema, or hypotension, are typically treated by rapid electrical cardioversion. Patients thus cardioverted to a normal sinus rhythm can be treated with antiarrhythmic drugs, such as dofetilide, a class III antiarrhythmic indicated for maintenance of normal sinus rhythm in patients with atrial fibrillation of greater than one week duration who have been converted to normal sinus rhythm. (See TIKOSYN package insert, rev. 2014, which is hereby incorporated by reference herein in its entirety.) Treatment with dofetilide is sometimes accompanied by serious side effects. These include ventricular arrhythmias (including ventricular fibrillation, ventricular tachycardia and torsade de Pointes) and various forms of block (AV block, bundle branch block and heart block).

The incidence of these side effects appears related to QT prolongation, which itself is directly related to dofetilide plasma concentration. Clearance of dofetilide decreases with decreasing creatine clearance. Therefore, dosage adjustments are typically based on calculated creatine clearance. Accordingly, one way to obviate these life-threatening side effects is to administer an intravenous formulation of dofetilide only to those patients without ventricular arrhythmias or heart blocks. In embodiments, an intravenous formulation of dofetilide is administered to patients who have normal kidney function. In other embodiments, an intravenous formulation of dofetilide may be administered to patients with reduced kidney function. In embodiments, the patient has a creatinine clearance of about 20 mL/min to about 90 mL/min, such as about 40 mL/min to about 60 mL/min.

Such therapy can account for the black box warning given for TIKOSYN. Initiation of oral dofetilide therapy typically requires that patients be placed for a minimum of three days in a facility that can provide calculated creatinine clearance, continuous ECG monitoring, and cardiac resuscitation. Treatment with the intravenous formulation of dofetilide is expected to shorten this observation period to one day or less. This will result in substantial savings in both time and cost, while providing practitioners with all the necessary information relating to the patient's ability to leave the facility on oral dofetilide or another therapeutic agent.

The following Examples are provided to illustrate various protocols for administering anti-arrhythmics, such as dofetilide. Although dofetilide is primarily exemplified in the Examples, other anti-arrhythmics can be substituted, and depending on the particular anti-arrhythmic selected (such as for sotalol, amiodarone, ibutilide, dronedarone, procainamide, flecainide, or propafenone), the dose and/or rate of administration in any of the Examples can be adjusted up or down to be commensurate with the pharmacokinetics of the particular drug being administered.

### Example 1

An example dofetilide or anti-arrhythmic treatment protocol for a patient experiencing a ventricular conduction abnormality, such as atrial flutter, is described herein. The male patient, age 60, is admitted to the hospital to initiate anti-arrhythmic, such as dofetilide, treatment. The patient's creatinine clearance is measured/determined to be >60 mL/min. The patient is connected to an electrocardiograph, and treatment is initiated with an IV loading dose of dofetilide in an amount of up to 3.5 μg/kg infused over up to 1 hour. The patient's QT is measured/determined prior to start of the IV and is monitored every 15 minutes during the IV loading dose administration along with heart rate (HR) and blood pressure (BP). The patient's QTc is calculated from the QT measurement. If the patient's baseline (i.e., prior to dofetilide administration) QT interval or baseline QTc is greater than 500 msec (in patients with ventricular conduction abnormalities), then dofetilide is contraindicated. Measure every 15 min. during the IV loading dose, and if the QT or QTc increases by greater than 20% of the patient's baseline QT or baseline QTc (or is greater than 550 msec), then the IV dofetilide is discontinued or a subsequent lower dose is considered or administered. If after any subsequent IV or oral dose the QT or QTc increases to greater than 550 msec, then dofetilide is discontinued.

Immediately or one hour after completion of the loading dose, the patient receives a maintenance dose of dofetilide delivered by way of a second IV infusion of up to 2.5 μg/kg over 5 hours. The patient's QT or QTc is monitored every hour during the administration of the IV maintenance dose. If during or after the IV maintenance dose the patient's QT or QTc is greater than 550 msec or if the QT or QTc increases by greater than 20% of the patient's baseline QT or QTc, then the IV maintenance dose is reduced and subsequent IV maintenance doses are delivered at approximately 1.25 μg/kg over up to 5 hours.

For the situation where QT or QTc is within an acceptable range, one or more subsequent IV or oral maintenance doses, selected from a 2.5 μg/kg IV infusion over up to 5 hours or a 500 μg oral dose, are repeated every 12 hours until Cmax ss is reached and the patient can be released to continue dofetilide treatment orally outside the hospital. For the situation where (i) the patient's QT or QTc after administration of the first IV maintenance dose is greater than 550 msec, (ii) the patient's subsequent dose is reduced, and (iii) the QT or QTc for the subsequent reduced maintenance dose (or any subsequent maintenance dose) is observed to be greater than 550 msec, additional subsequent maintenance doses can be further reduced to a 0.5-0.9 μg/kg IV infusion over 5 hours, and/or given over a longer infusion time, and/or reduced to a 125 μg oral dose, or dofetilide treatment is discontinued.

### Example 2

An example anti-arrhythmic, such as dofetilide, treatment protocol for a patient experiencing a ventricular conduction abnormality, such as atrial flutter, is described herein. The male patient, age 60, is admitted to the hospital to initiate treatment. The patient's creatinine clearance is determined to be between 40 and 60 mL/min. The patient is connected to an electrocardiograph, and an initial (or baseline) QT interval is determined to be not greater than 500 msec. Treatment is initiated with an IV loading dose of up to 1.6 μg/kg infused over up to 1 hour. The loading dose may be administered by way of several smaller IV doses. The patient's QT interval is measured every 15 minutes during the IV loading dose administration along with HR and BP.

During or following administration of the IV loading dose, the patient's QT interval is determined to be greater than 550 msec. A reduced maintenance dose is administered to the patient up to 6 hours after completion of the IV loading dose or dofetilide administration is discontinued. The reduced maintenance dose is administered as an oral dose of 125 μg. The patient's QT interval is monitored following administration of the oral maintenance dose. The patient's QT interval no longer exceeds 550 msec and the ΔQTc is not greater than 20% of the patient's initial QTc. Subsequent oral maintenance doses of 125 μg are given every 12 to 48 hours.

### Example 3

An example anti-arrhythmic/dofetilide treatment protocol for a patient experiencing a ventricular conduction abnormality, such as atrial flutter, is described herein. The male patient, age 60, is admitted to the hospital to initiate treatment. The patient's creatinine clearance is determined to be between 20 and 40 mL/min. The patient is connected to an electrocardiograph, and an initial QT interval is determined to be not greater than 500 msec. Treatment is initiated with an IV loading dose of up to 1.6 μg/kg infused over up to 1 hour. The loading dose may be administered by way of several smaller IV doses. The patient's QT interval is measured every 15 minutes during the IV loading dose administration along with HR and BP.

During and following administration of the loading dose, the patient's QT interval is determined to be not greater than 550 msec and ΔQTc is not greater than 20% of the patient's initial QTc. A maintenance dose is administered to the patient up to 6 hours after completion of the IV loading dose. The maintenance dose is administered as an oral dose of 125 μg. The patient's QT interval is monitored following administration of the oral maintenance dose. The patient's QT interval still does not exceed 550 msec and is not greater than 20% of the patient's initial QTc. Subsequent oral maintenance doses of 125 μg are given every 12 to 48 hours.

### Example 4

An example anti-arrhythmic/dofetilide treatment protocol for a patient experiencing a ventricular conduction abnormality, such as atrial flutter, is described herein. The male patient, age 60, is admitted to the hospital to initiate treatment. The patient's creatinine clearance is determined to be >60 mL/min. The patient is connected to an electrocardiograph, and an initial QT interval is determined. Treatment is initiated with an IV loading dose based on an oral target dose of 500 μg, which is administered in an amount of up to 3 μg/kg and is infused over up to 1 hour. The patient's QT interval is measured every 15 minutes during the IV loading dose administration along with HR and BP.

During and following administration of the loading dose, the patient's QT interval is determined to be not greater than 550 msec and the ΔQTc is not greater than 20% of the patient's initial QTc. A maintenance dose is administered to the patient as an IV dose of up to 3 μg/kg. The IV maintenance dose is administered over a time period of up to 60 minutes. The patient's QT interval is monitored during and following administration of the IV maintenance dose. The patient's QT interval still does not exceed 550 msec and the ΔQTc is not greater than 20% of the patient's initial QTc. Subsequent oral maintenance doses of 500 μg are given every 12 to 48 hours.

### Example 5

An example anti-arrhythmic/dofetilide treatment protocol for a patient experiencing a ventricular conduction abnormality, such as atrial flutter, is described herein. The male patient, age 60, is admitted to the hospital to initiate treatment. The patient's creatinine clearance is determined to be between 40 and 60 mL/min. The patient is connected to an electrocardiograph, and an initial QT interval is determined. Treatment is initiated with an IV loading dose based on an oral target of 250 μg, which is administered in an amount of up to 1.6 μg/kg and is infused over about 1 hour. The patient's QT interval is measured every 15 minutes during the IV loading dose administration along with HR and BP.

During and following administration of the loading dose, the patient's QT interval is determined to be not greater than 550 msec and the ΔQTc is not greater than 20% of the patient's initial QTc. A maintenance dose is administered to the patient as an IV dose of up to 1.6 μg/kg. The IV dose is administered over a time period of up to 60 minutes. The patient's QT interval is monitored during and following administration of the IV maintenance dose. The patient's QT interval still does not exceed 550 msec and the ΔQTc is not greater than 20% of the patient's initial QTc. Subsequent IV maintenance doses of up to 1.6 μg/kg are given every 12 to 48 hours.

### Example 6

An example anti-arrhythmic/dofetilide treatment protocol for a patient experiencing a ventricular conduction abnormality, such as atrial flutter, is described herein. The male patient, age 60, is admitted to the hospital to initiate treatment. The patient's creatinine clearance is determined to be between 20 and 40 mL/min. The patient is connected to an electrocardiograph, and an initial QT interval is determined. Treatment is initiated with an IV loading dose based on an oral target of 125 μg, which is administered in an amount of up to 0.8 μg/kg and is infused over about 1 hour. The patient's QT interval is measured every 15 minutes during the IV loading dose administration along with HR and BP.

During and following administration of the IV loading dose, the patient's QT interval is determined to be not greater than 550 msec and the ΔQTc is not greater than 20% of the patient's initial QTc. A maintenance dose is administered to the patient as an IV dose of up to 0.8 μg/kg. The IV maintenance dose is administered over a time period of up to 60 minutes. The patient's QT interval is monitored during and following administration of the IV maintenance dose. The patient's QT interval still does not exceed 550 msec and the ΔQTc is not greater than 20% of the patient's initial QTc. Subsequent IV maintenance doses of up to 0.8 μg/kg are given every 12 to 48 hours.

### Example 7

An example anti-arrhythmic/dofetilide treatment protocol for a patient experiencing a ventricular conduction abnormality, such as atrial flutter, is described herein. The female patient, age 60, is admitted to the hospital to initiate treatment. The patient's creatinine clearance is determined to be >60 mL/min. The patient is connected to an electrocardiograph, and an initial QT interval is determined to be in an acceptable range. Treatment is initiated with an IV loading dose in the range of 1.6-3 μg/kg. The patient's QT interval is measured every 15 minutes during the IV loading dose administration along with HR and BP.

During or following administration of the IV loading dose, the patient's QT interval is determined to be not greater than 550 msec, but the ΔQTc is greater than 20% of the patient's initial QTc. A maintenance dose is administered to the patient in a reduced amount as an oral dose of 250 μg. The patient's QT interval is monitored following administration of the first oral maintenance dose. The patient's QT interval still does not exceed 550 msec and the ΔQTc is no longer greater than 20% of the patient's initial QTc. Subsequent oral maintenance doses of 250 μg are given every 12 to 48 hours.

### Example 8

An example anti-arrhythmic/dofetilide treatment protocol for a patient experiencing a ventricular conduction abnormality, such as atrial flutter, is described herein. The male patient, age 50, is admitted to the hospital to initiate treatment. The patient's creatinine clearance is determined to be between 40 and 60 mL/min. The patient is connected to an electrocardiograph, and an initial QT interval is determined. Treatment is initiated with an IV loading dose of 0.8-1.6 μg/kg. The patient's QT interval is measured every 15 minutes during the IV loading dose administration along with HR and BP.

During and following administration of the IV loading dose, the patient's QT interval is determined to be not greater than 550 msec and the ΔQTc is not greater than 20% of the patient's initial QTc. A first maintenance dose is administered to the patient as an oral dose of 250 μg. The patient's QT interval is monitored following administration of the first oral maintenance dose. The patient's QT interval is determined to be less than 550 msec and the ΔQTc is not greater than 20% of the patient's initial QTc. Subsequent oral maintenance doses of 250 μg are given every 12 to 48 hours.

### Example 9

An example anti-arrhythmic/dofetilide treatment protocol for a patient experiencing a ventricular conduction abnormality, such as atrial flutter, is described herein. The male patient, age 40, is admitted to the hospital to initiate treatment. The patient's creatinine clearance is determined to be between 20 and 40 mL/min. The patient is connected to an electrocardiograph, and an initial QT interval is determined. Treatment is initiated with an IV loading dose of 0.1-0.8 μg/kg. The patient's QT interval is measured every 15 minutes during the IV loading dose administration along with HR and BP.

During and following administration of the IV loading dose, the patient's QT interval is not greater than 550 msec and the ΔQTc is not greater than 20% of the patient's initial QTc. A first maintenance dose is administered to the patient as an oral dose of 125 μg. The patient's QT interval is monitored following administration of the first oral maintenance dose. The patient's QT interval is determined to be less than 550 msec and the ΔQTc is not greater than 20% of the patient's initial QTc. Subsequent oral maintenance doses of 125 μg are given every 12 to 48 hours.

### Example 10

An example anti-arrhythmic/dofetilide treatment protocol for a patient experiencing a ventricular conduction abnormality, such as atrial flutter, is described herein. The female patient, age 40, is admitted to the hospital to initiate treatment. The patient's creatinine clearance is determined to be between >60 mL/min. The patient is connected to an electrocardiograph, and an initial QT interval is determined. Treatment is initiated with an IV loading dose of about 3 μg/kg. The patient's QT interval is measured every 15 minutes during the IV loading dose administration along with HR and BP.

During or following administration of the IV loading dose, the patient's QT interval is determined to be greater than 550 msec. A first maintenance dose is administered to the patient as an oral dose in the reduced amount of 250 μg. The patient's QT interval is monitored following administration of the first oral maintenance dose. The patient's QT interval is still determined to be greater than 550 msec. Subsequent oral maintenance doses of 125 μg are given every 12 to 48 hours or dofetilide administration is discontinued.

### Example 11

An example anti-arrhythmic/dofetilide treatment protocol for a patient experiencing a ventricular conduction abnormality, such as atrial flutter, is described herein. The female patient, age 40, is admitted to the hospital to initiate treatment. The patient's creatinine clearance is determined to be between 40 to 60 mL/min. The patient is connected to an electrocardiograph, and an initial QT interval is determined. Treatment is initiated with an IV loading dose of about 1.6 μg/kg. The patient's QT interval is measured every 15 minutes during the IV loading dose administration along with HR and BP.

During and following administration of the IV loading dose, the patient's QT interval is determined to be 550 msec or less and the ΔQTc is not greater than 20% of the patient's initial QTc. A first maintenance dose is administered to the patient as an oral dose of 250 μg. The patient's QT interval is monitored following administration of the first oral maintenance dose. The patient's QT interval is still determined to be less than 550 msec and the ΔQTc is not greater than 20% of the patient's initial QTc. The physician would like to further lengthen the patient's QTc, therefore subsequent oral maintenance doses of 500 μg are given every 12 to 48 hours.

### Example 12

An example anti-arrhythmic/dofetilide treatment protocol for a patient experiencing a ventricular conduction abnormality, such as atrial flutter, is described herein. The male patient, age 40, is admitted to the hospital to initiate treatment. The patient's creatinine clearance is determined to be between 20 and 40 mL/min. The patient is connected to an electrocardiograph, and an initial QT interval is determined. Treatment is initiated with an IV loading dose in an amount ranging from 0.1-0.8 μg/kg. The patient's QT interval is measured every 15 minutes during the IV loading dose administration along with HR and BP.

During and following administration of the IV loading dose, the patient's QT interval is not greater than 550 msec and the ΔQTc is not greater than 20% of the patient's initial QTc. A first maintenance dose is administered to the patient as an oral dose of 125 μg. The patient's QT interval is monitored following administration of the first oral maintenance dose and the patient's QT interval is determined to be not greater than 550 msec, and the ΔQTc is not greater than 20% of the patient's initial QTc. A second maintenance dose is administered to the patient as an oral dose of 125 μg and is given 12 to 24 hours after the first maintenance dose. The patient's QTc interval is monitored following administration of the second oral maintenance dose and the patient's QTc interval is determined to be not greater than 550 msec, but the ΔQTc is greater than 20% of the patient's initial QTc (or the patient's QTc interval is determined to be greater than 550 msec, but the ΔQTc is not greater than 20% of the patient's initial QTc, or both are outside of the acceptable range). Subsequent oral maintenance doses of 125 μg are given farther apart and every 24 to 48 hours.

### Example 13

An example anti-arrhythmic/dofetilide treatment protocol for a patient currently being administered oral dofetilide for treatment of a ventricular conduction abnormality, such as atrial flutter, is described herein. The male patient, age 60, is admitted to the hospital to uptitrate dofetilide treatment from a 250 μg BID protocol to a 500 μg BID protocol. The patient's creatinine clearance is determined to be >60 mL/min. The patient is connected to an electrocardiograph, and an initial QTc interval is determined. An IV loading dose is administered to the patient based on an oral target dose of 500 μg, which is administered in an amount of up to 3 μg/kg and is infused over up to 1 hour. The patient's QTc interval is measured every 15 minutes during the IV loading dose administration along with HR and BP.

During and following administration of the loading dose, the patient's QTc interval is determined not to exceed 550 msec and the ΔQTc is not greater than 20% of the patient's initial QTc. A maintenance dose is administered to the patient as an IV dose of up to 3 μg/kg. The IV maintenance dose is administered over a time period of up to 60 minutes. The patient's QTc interval is monitored during and following administration of the IV maintenance dose. The patient's QTc interval still does not exceed 550 msec and the ΔQTc is not greater than 20% of the patient's initial QTc. Subsequent oral maintenance doses of 500 μg are given every 12 hours.

The present disclosure has described particular implementations having various features. In light of the disclosure provided above, it will be apparent to those skilled in the art that various modifications and variations can be made without departing from the scope or spirit of the disclosure. One skilled in the art will recognize that the disclosed features may be used singularly, in any combination, or omitted based on the requirements and specifications of a given application or design. When an implementation refers to “comprising” certain features, it is to be understood that the implementations can alternatively “consist of” or “consist essentially of” any one or more of the features. Other implementations will be apparent to those skilled in the art from consideration of the specification and practice of the disclosure.

It is noted in particular that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the disclosure fall within the scope of the disclosure. Further, all of the references cited in this disclosure including patents, published applications, and non-patent literature are each individually incorporated by reference herein in their entireties and as such are intended to provide an efficient way of supplementing the enabling disclosure as well as provide background detailing the level of ordinary skill in the art.

1. A method, comprising:
administering dofetilide to a patient by way of an intravenous loading dose over a duration of at least 1 hour and in an amount effective for treating or preventing a cardiovascular condition of the patient.

2. The method of claim 1, wherein the amount of dofetilide is in the range of about ±50% of a target maintenance dose.

3. The method of claim 2, wherein the loading dose is selected from an amount in the range of about ±25% of the amount of a target maintenance dose.

4. The method of claim 1, wherein the cardiovascular condition is selected from atrial fibrillation, atrial flutter, ventricular tachycardia, hemodynamically stable or unstable ventricular tachycardia, paroxysmal atrial fibrillation, ventricular fibrillation, paroxysmal supraventricular tachycardia, heart failure, coronary artery disease, pulmonary artery hypertension, atrial tachycardia, junctional ectopic tachycardia, or junctional tachycardia.

5. The method of claim 1, further comprising measuring a creatinine clearance of the patient before the administering, and selecting or adjusting the effective amount based on the creatinine clearance.

6. The method of claim 5, wherein the effective amount is selected from:
i. 1.6-3 μg/kg, when the creatinine clearance of the patient is >60 mL/min;
ii. 0.8-1.6 μg/kg, when the creatinine clearance of the patient is 40 to 60 mL/min; or
iii. 0.1-0.8 μg/kg, when the creatinine clearance of the patient is 20 to <40 mL/min.

7. The method of claim 1, further comprising administering to the patient one or more IV maintenance dose of dofetilide selected from an amount ranging from 0.1-10 μg/kg.

8. The method of claim 7, wherein the subject is unable (NPO) to take oral dofetilide.

9. The method of claim 1, further comprising administering one or more oral dose of dofetilide to the patient.

10. The method of claim 1, wherein the amount of the IV loading dose is higher than 3 μg/kg.

11. The method of claim 10, wherein the patient has a creatinine clearance of 20-60 mL/min.

12. A method, comprising:
administering dofetilide to a subject with atrial fibrillation cardioverted to normal sinus rhythm; and
wherein the dofetilide is administered intravenously as a loading dose over a duration of at least 1 hour and in an amount effective for maintenance of normal sinus rhythm.

13. The method of claim 12, wherein the loading dose is selected from an amount in the range of about ±50% of the amount of a maintenance dose for the subject.

14. The method of claim 13, wherein the loading dose is selected from an amount in the range of about ±25% of the amount of the maintenance dose.

15. A method of initiating or escalating dofetilide treatment in a patient, comprising:
(A) determining a first QT interval of the patient;
(B) administering to the patient an IV loading dose of dofetilide over at least 1 hour;
(C) administering a first oral dose of dofetilide to the patient after completion of the IV loading dose, wherein the first oral dose is chosen from 125 μg, 250 μg, or 500 μg;
(D) determining a second QT interval of the patient; and
(E) administering at least one subsequent oral dose of dofetilide to the patient;
wherein the amount of the IV loading dose is in the range of about ±50% of the first oral dose and/or one or more of the subsequent oral doses.

16. The method of claim 15, wherein:
the one or more subsequent oral doses is a higher dose than the first oral dose to further increase the second QT interval; or
the one or more subsequent oral doses is a lower dose than the first oral dose to reduce a subsequent QT interval of the patient.

17. The method of claim 15, wherein the dofetilide is administered to the patient for treatment of or prevention of atrial fibrillation or atrial flutter.

18. The method of claim 15, further comprising measuring a creatinine clearance of the patient before administering the IV loading dose, and selecting or adjusting an amount thereof based on the creatinine clearance.

19. The method of claim 15, wherein the amount of the IV loading dose is greater than 3 μg/kg.
